(19) United States
(12) Patent Application Publication
(10) Pub. No.: US 2025/0162963 A1
(43) Pub. Date: May 22, 2025

(54) ETHYLENE TRIMERIZATION CATALYST SYSTEMS CONTAINING AROMATIC SOLVENTS

(71) Applicant: Chevron Phillips Chemical Company LP, The Woodlands, TX (US)

(72) Inventors: Steven M. Bischof, Kingwood, TX (US); Pasquale Iacono, Bartlesville, OK (US); Brooke L. Small, Kingwood, TX (US); Orson L. Sydora, Kingwood, TX (US); Sean K. McLaughlin, Kingwood, TX (US); Bruce E. Kreischer, Kingwood, TX (US)

(21) Appl. No.: 18/944,112

(22) Filed: Nov. 12, 2024

Related U.S. Application Data
(60) Provisional application No. 63/600,067, filed on Nov. 17, 2023.

(51) Int. Cl. C07C 2/32; B01J 31/02; B01J 31/14; B01J 31/22

(52) U.S. Cl. CPC C07C 2/32; B01J 31/0244; B01J 31/143; B01J 31/2226; B01J 2231/20; B01J 2531/31; B01J 2531/62; C07C 2531/14; C07C 2531/22

(57) ABSTRACT

Catalyst compositions containing a pyrrole compound, a chromium compound, an organoaluminum compound, and an aromatic hydrocarbon solvent comprising xylenes or C₉ substituted benzenes are disclosed. Related ethylene oligomerization processes utilizing the catalyst compositions to produce oligomer products containing 1-hexene also are described.

### REFERENCE TO RELATED APPLICATION

This application claims the benefit of U.S. Provisional Patent Application No. 63/600,067, filed on Nov. 17, 2023, the disclosure of which is incorporated herein by reference in its entirety.

### FIELD OF THE INVENTION

The present disclosure relates generally to a catalyst composition containing a pyrrole compound, a chromium compound, an organoaluminum compound, and an aromatic hydrocarbon solvent comprising xylenes or C9 substituted benzenes, and the use of the catalyst composition in an ethylene oligomerization process.

### BACKGROUND OF THE INVENTION

Alpha olefins such as 1-hexene can be produced using an ethylene reactant and various combinations of catalyst systems and oligomerization processes. It can be beneficial for the catalyst system employed to have high catalytic activity and good compatibility with other materials present in the oligomerization reactor, as well as being selective to desirable C₆ linear α-olefins with minimal amounts of undesirable contaminants in the 1-hexene product stream. Accordingly, it is to these ends that the present invention is generally directed.

### SUMMARY OF THE INVENTION

This summary is provided to introduce a selection of concepts in a simplified form that are further described herein. This summary is not intended to identify required or essential features of the claimed subject matter. Nor is this summary intended to be used to limit the scope of the claimed subject matter.

Disclosed herein are catalyst compositions and methods for using the catalyst compositions to oligomerize ethylene. In one aspect, the catalyst composition can comprise (a) a pyrrole compound, (b) a chromium compound, (c) an organoaluminum compound, and (d) an aromatic hydrocarbon solvent comprising xylenes. In another aspect, the catalyst composition can comprise (A) a pyrrole compound, (B) a chromium compound, (C) an organoaluminum compound, and (D) an aromatic hydrocarbon solvent comprising C₉ substituted benzenes.

A representative oligomerization process encompassed herein can comprise (i) contacting ethylene, an organic reaction medium, optionally hydrogen, and any of the catalyst compositions disclosed herein in an oligomerization reactor, (ii) forming an oligomer product in the oligomerization reactor, the oligomer product comprising hexenes and higher oligomers, and (iii) discharging an effluent stream from the oligomerization reactor, the effluent stream comprising unreacted ethylene and the oligomer product.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and are included to further demonstrate certain aspects of the present invention. The invention may be better understood by reference to these figures in combination with the detailed description.

FIG. 1 is a bar chart of the 1-hexene productivity (kg/g Cr) and C₁₀ wt. % at a 3.4 ppm reactor concentration of chromium as a function of the aromatic hydrocarbon solvent utilized in Examples 1-8.

FIG. 2 is a bar chart of the 1-hexene productivity (kg/g Cr) and C₁₀ wt. % at a 1.5 ppm reactor concentration of chromium as a function of the aromatic hydrocarbon solvent utilized in Examples 1-8.

FIG. 3 is a bar chart of the C₆ productivity (g C₆/g Cr) as a function of the aromatic hydrocarbon solvent utilized in Comparative Example 1 (ethylbenzene) and Example 2 (xylenes).

FIG. 4 is a bar chart of the C₆ product purity (wt. %) as a function of the aromatic hydrocarbon solvent utilized in Comparative Example 1 (ethylbenzene) and Example 2 (xylenes).

FIG. 5 is a bar chart of the C₁₀ byproduct (wt. %) as a function of the aromatic hydrocarbon solvent utilized in Comparative Example 1 (ethylbenzene) and Example 2 (xylenes).

FIG. 6 is a bar chart of the C₈ and C₁₂ byproducts (wt. %) as a function of the aromatic hydrocarbon solvent utilized in Comparative Example 1 (ethylbenzene) and Example 2 (xylenes).

While the inventions disclosed herein are susceptible to various modifications and alternative forms, only a few specific aspects have been shown by way of example in the drawings and described in detail below. The figures and detailed description of specific aspects are not intended to limit the breadth or scope of the inventive concepts or the appended claims in any manner. Rather, the figures and detailed description are provided to illustrate the inventive concepts to a person of ordinary skill in the art and to enable such person to make and use the inventive concepts.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compounds, compositions, processes, or methods described herein are contemplated with or without explicit description of the particular combination. Additionally, unless explicitly recited otherwise, any aspect or feature disclosed herein can be combined to describe inventive compounds, compositions, processes, or methods consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. For example, a catalyst composition consistent with aspects of the present invention can comprise; alternatively, can consist essentially of; or alternatively, can consist of; a pyrrole compound, a chromium compound, an organoaluminum compound, and an aromatic hydrocarbon solvent. The terms “a,” “an,” and “the” are intended to include plural alternatives, e.g., at least one, unless otherwise specified. For instance, the disclosure of “an organoaluminum compound” is meant to encompass one, or mixtures or combinations of two or more organoaluminum compounds, unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any generic or specific compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to pentane includes n-pentane, 2-methyl-butane, and 2,2-dimethylpropane; and a general reference to a butyl group includes an n-butyl group, a sec-butyl group, an iso-butyl group, and a t-butyl group. Likewise, a general reference to hexenes includes all linear or branched, acyclic or cyclic, hydrocarbon compounds having six carbon atoms and one carbon-carbon double bond. Thus, hexenes encompass, for instance, 1-hexene, 2-hexene, 3-hexene, neohexene, and cyclohexene. When a stream or product/composition is indicated to contain a certain amount of hexenes, this amount represents the total amount of hexenes present, whether only a single compound (e.g., 1-hexene) or a mixture of different compounds in any relative proportion. A similar interpretation applies for xylenes (including ortho-xylene, meta-xylene, para-xylene, or mixtures thereof).

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon. Non-limiting examples of hydrocarbyl groups include alkyl, alkenyl, aryl, and aralkyl groups, amongst other groups.

The term “oligomer” refers to a compound that contains from 2 to 20 monomer units. The terms “oligomerization product” and “oligomer product” include all products made by the “oligomerization” process, including the oligomers and products which are not oligomers (e.g., products which contain more than 20 monomer units, or solid polymer), but exclude other non-oligomer components of an oligomerization reactor effluent stream, such as unreacted ethylene, organic reaction medium, and hydrogen, amongst other components.

The terms “catalyst composition,” “catalyst mixture,” “catalyst system,” and the like, do not depend upon the actual product or composition resulting from the contact or reaction of the initial components of the disclosed or claimed catalyst composition (or catalyst mixture or catalyst system), the nature of the active catalytic site, or the fate of the pyrrole compound, the chromium compound, the organoaluminum compound, and the aromatic hydrocarbon solvent after combining these components. Therefore, the terms “catalyst composition,” “catalyst mixture,” “catalyst system,” and the like, encompass the initial starting components of the composition, as well as whatever products may result from contacting these initial starting components, and this is inclusive of both heterogeneous and homogenous catalyst systems or compositions. The terms “catalyst composition,” “catalyst mixture,” “catalyst system,” and the like, may be used interchangeably throughout this disclosure.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, the molar ratio of chromium:aluminum in the catalyst composition can be in various ranges. By a disclosure that the molar ratio can range from 1:1 to 1:150, the intent is to recite that the molar ratio can be any ratio within the range and, for example, can include any range or combination of ranges from 1:1 to 1:150, such as from 1:1 to 1:100, from 1:3 to 1:50, or from 1:9 to 1:21, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

Disclosed herein are catalyst compositions containing a pyrrole compound, a chromium compound, an organoaluminum compound, and an aromatic hydrocarbon solvent comprising xylenes or C₉ substituted benzenes, and ethylene oligomerization processes utilizing the catalyst compositions to selectively produce 1-hexene. Advantageously and unexpectedly, by utilizing an aromatic hydrocarbon solvent comprising xylenes or C₉ substituted benzenes as a substitute for an ethylbenzene solvent when preparing the disclosed catalyst compositions, no significant differences are observed in 1-hexene product purity, productivity, and polymer byproduct formation. Therefore, with these catalyst solvent alternatives, the overall process is improved and simplified by reducing the benzene content in the hexene product stream and eliminating the need for pre-distillation of ethylbenzene solvents to reduce the benzene content prior to use in catalyst preparation.

An objective of this invention is to develop an improved ethylene oligomerization process by utilizing a low benzene aromatic hydrocarbon solvent comprising xylenes or C₉ substituted benzenes in the preparation of the oligomerization catalyst system. Another objective of this invention is to develop catalyst systems that provide excellent catalytic activity in ethylene oligomerization processes, resulting in high yields of oligomer products, as well as being highly selective in the formation of desirable C₆ linear α-olefins.

### Catalyst Compositions

A first catalyst composition consistent with aspects of this invention can contain (a) a pyrrole compound, (b) a chromium compound, (c) an organoaluminum compound, and (d) an aromatic hydrocarbon solvent comprising xylenes. A second catalyst composition consistent with aspects of this invention can comprise (A) a pyrrole compound, (B) a chromium compound, (C) an organoaluminum compound, and (D) an aromatic hydrocarbon solvent comprising C₉ substituted benzenes.

Generally, the pyrrole compound utilized as a component in the catalyst compositions and oligomerization processes disclosed herein can be any suitable pyrrole compound or any pyrrole compound disclosed herein. A pyrrole compound is defined as a compound comprising a 5-membered, nitrogen-containing heterocycle, such as for example, pyrrole, derivatives of pyrrole, and mixtures thereof. As used in this disclosure, the term “pyrrole compound” refers to pyrrole (C₅H₅N), derivatives of pyrrole (e.g., indole), substituted pyrroles, as well as metal pyrrolide complexes. Broadly, the pyrrole compound can be pyrrole or any heteroleptic or homoleptic metal complex or salt containing a pyrrolide radical or ligand. In a nonlimiting aspect, pyrroles include hydrogen pyrrole, derivatives of pyrrole, substituted pyrrole, lithium pyrrole, sodium pyrrole, potassium pyrrole, cesium pyrrole, the salts of a substituted pyrrole, or combinations thereof. In an aspect, the pyrrole compound can be characterized as having formula (A), wherein R, R¹, R², and R³ independently can be H or a C₁ to C₃₆ hydrocarbyl group:

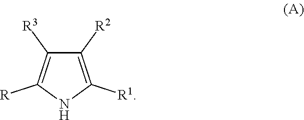

Formula (A) is not designed to show stereochemistry or isomeric positioning of the different moieties (e.g., this formula is not intended to show cis or trans isomers), although such compounds are contemplated and encompassed by this formula. Substituents R, R¹, R², and R³ are described herein and can be utilized without limitation to describe aspects of a pyrrole compound having formula (A). In this formula, R, R¹, R², and R³ independently can be H or a C₁ to C₃₆ hydrocarbyl group, and it is contemplated that any of R, R¹, R², and R³ can be the same or different.

In one aspect, for instance, R, R¹, R², and R³ independently can be H or a C₁ to C₁₈ hydrocarbyl group. In another aspect, R, R¹, R², and R³ independently can be H or a C₁ to C₁₂ hydrocarbyl group. In yet another aspect, R, R¹, R², and R³ independently can be H or a C₁ to C₈ hydrocarbyl group. In still another aspect, R, R¹, R², and R³ independently can be H or a C₁ to C₄ hydrocarbyl group. Any hydrocarbyl group, independently, can be an alkyl group, a cycloalkyl group, an aryl group (e.g., a phenyl group or a naphthyl group, optionally substituted), or an aralkyl group (e.g., a benzyl group, optionally substituted).

In an aspect, R, R¹, R², and R³ independently can be H, a methyl group, an ethyl group, a propyl group (n-propyl group or iso-propyl group), a butyl group (n-butyl group, iso-butyl group, sec-butyl group, or tert-butyl group), a pentyl group (n-pentyl group, iso-pentyl group, sec-pentyl group, or neopentyl group), a hexyl group, a heptyl group, an octyl group, a nonyl group, a decyl group, a cyclobutyl group, a cyclopentyl group, a cyclohexyl group, a cycloheptyl group, an adamantyl group, a phenyl group, a naphthyl group, or a benzyl group. For instance, R, R¹, R², and R³ independently can be H or a methyl group (or an ethyl group). Alternatively, R, R¹, R², and R³ independently can be, in some aspects, H, a methyl group, an ethyl group, a propyl group (n-propyl group or iso-propyl group), a butyl group (n-butyl group, iso-butyl group, sec-butyl group, or tert-butyl group), a cyclohexyl group, an adamantyl group, or a phenyl group.

In an aspect, the pyrrole compound can comprise, consist essentially of, or consist of, a pyrrole compound having a C₁ to C₁₈ group attached to the 2- and 5-positions of the pyrrole. Unless otherwise specified, the pyrrole compound having a C₁ to C₁₈ group attached to the 2- and 5-positions can have groups attached at the 3 and/or 4 positions. In an aspect, the pyrrole compound of the oligomerization catalyst system can be a 2,5-disubstituted pyrrole compound, that is, the pyrrole compound has substituents only at the 2- and 5-positions. Regardless of whether or not the pyrrole compound has substituents present at the 3 and/or 4 positions, the groups attached to the 2- and 5-positions of the pyrrole compound may be the same or different. For example, 2,5-dimethyl pyrrole, 2,5-diethyl pyrrole, 2-ethyl-5-methyl pyrrole, and 2-ethyl-5-propyl pyrrole are among the suitable 2,5-disubstituted pyrroles for use in the catalyst systems and oligomerization processes of this disclosure. Generally, the groups attached to the 2- and 5-position of the pyrrole compound can be any pyrrole substituent group disclosed herein. Additional examples of pyrroles include, but are not limited to, pyrrole-2-carboxylic acid, 2-acetylpyrrole, pyrrole-2-carboxaldehyde, tetrahydroindole, 2,4-dimethyl-3-ethylpyrrole, 3-acetyl-2,4-dimethylpyrrole, ethyl-2,4-dimethyl-5-(ethoxycarbonyl)-3-pyrrole-propionate, and ethyl-3,5-dimethyl-2-pyrrole-carboxylate, and the like. In a particular aspect of this invention, the pyrrole compound comprises 2,5-dimethylpyrrole, 2,5-diethylpyrrole, or both.

The chromium compound utilized as a component in the catalyst compositions and oligomerization processes disclosed herein can be any suitable chromium compound or any chromium compound disclosed herein. The chromium compound can have a chromium oxidation state of 0 to 6. In some aspects, the chromium within the chromium compound can have an oxidation state of 2 or 3 (i.e., a chromium(II) or chromium(III) compound). In other aspects, the chromium within the chromium compound can have an oxidation state of 2 (i.e., a chromium(II) compound); or alternatively, have an oxidation state of 3 (i.e., a chromium(III) compound). For example, chromium(II) compounds which can be used as the chromium compound for the oligomerization catalyst system can comprise, consist essentially of, or consist of, chromium(II) nitrate, chromium(II) sulfate, chromium(II) fluoride, chromium(II) chloride, chromium(II) bromide, or chromium(II) iodide. Also by way of example, the chromium(III) compounds which can be used as the chromium compound for the oligomerization catalyst system may comprise, consist essentially of, or consist of, chromium(III) nitrate, chromium(III) sulfate, chromium(III) fluoride, chromium(III) chloride, chromium(III) bromide, or chromium(III) iodide. Alternatively, the chromium compounds that can be used as the chromium compound for the oligomerization catalyst system can comprise, consist essentially of, or consist of, chromium(II) nitrate; alternatively, chromium(II) sulfate; alternatively, chromium(II) fluoride; alternatively, chromium(II) chloride; alternatively, chromium(II) bromide; alternatively, chromium(II) iodide; alternatively, chromium(III) nitrate; alternatively, chromium(III) sulfate; alternatively, chromium(III) fluoride; alternatively, chromium(III) chloride; alternatively, chromium(III) bromide; or alternatively, chromium(III) iodide.

In yet an additional aspect of this disclosure, the chromium compound for the oligomerization catalyst system can comprise, consist essentially of, or consist of, a chromium(II) alkoxide, a chromium(II) carboxylate, a chromium(II) beta-dionate, a chromium(III) alkoxide, a chromium(III) carboxylate, or a chromium(III) beta-dionate; alternatively, a chromium(II) alkoxide or a chromium(III) alkoxide; alternatively, a chromium(II) carboxylate or a chromium(III) carboxylate; alternatively, a chromium(II) beta-dionate or a chromium(III) beta-dionate; alternatively, a chromium(II) alkoxide; alternatively, a chromium(II) carboxylate; alternatively, a chromium(II) beta-dionate; alternatively, a chromium(III) alkoxide; alternatively, a chromium(III) carboxylate; or alternatively, a chromium(III) beta-dionate.

In an aspect, each carboxylate group of the chromium compound can be a C₂ to C₂₄ carboxylate group; alternatively, a C₄ to C₁₉ carboxylate group; or alternatively, a C₅ to C₁₂ carboxylate group. In some aspects, each alkoxy group of the chromium compound can be a C₁ to C₂₄ alkoxy group; alternatively, a C₄ to C₁₉ alkoxy group; or alternatively, a C₅ to C₁₂ alkoxy group. In other aspects, each aryloxy group of the chromium compound can be a C₆ to C₂₄ aryloxy group; alternatively, a C₆ to C₁₉ aryloxy group; or alternatively, a C₆ to C₁₂ aryloxy group. In yet other aspects, each beta-dionate group of the chromium compound can be a C₅ to C₂₄ beta-dionate group; alternatively, a C₅ to C₁₉ beta-dionate group; or alternatively, a C₅ to C₁₂ beta-dionate group.

Chromium carboxylates are particularly useful chromium compounds for the oligomerization catalyst system. Thus, in one aspect, the catalyst system and process according to this disclosure provides for the use of chromium carboxylate compositions, including but are not limited to, chromium carboxylate compositions in which the carboxylate is a C₂ to C₂₄ monocarboxylate; alternatively, a C₄ to C₁₉ monocarboxylate; or alternatively, a C₅ to C₁₂ monocarboxylate. Some widely employed chromium carboxylate composition catalysts are those of chromium(III), for example, chromium(III) compositions comprising 2-ethylhexanoate are effective catalyst system components for selective 1-hexene synthesis.

In one aspect, the carboxylate group of the chromium carboxylate can be a C₂ to C₂₄ monocarboxylate. In an aspect, the carboxylate group of the chromium carboxylate may be an acetate, a propionate, a butyrate, a pentanoate, a hexanoate, a heptanoate, an octanoate, a nonanoate, a decanoate, an undecanoate, a dodecanoate, a tridecanoate, a tetradecanoate, a pentadecanoate, a hexadecanoate, a heptadecanoate, or an octadecanoate; or alternatively, a pentanoate, a hexanoate, a heptanoate, an octanoate, a nonanoate, a decanoate, a undecanoate, or a dodecanoate. In some aspects, the carboxylate group of the chromium carboxylate can be acetate, propionate, n-butyrate, isobutyrate, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, laurate (n-dodecanoate), or stearate (n-octadecanoate); alternatively, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, acetate; alternatively, propionate; alternatively, n-butyrate; alternatively, isobutyrate; alternatively, valerate (n-pentanoate); alternatively, neo-pentanoate; alternatively, capronate (n-hexanoate); alternatively, n-heptanoate; alternatively, caprylate (n-octanoate); alternatively, 2-ethylhexanoate; alternatively, n-nonanoate; alternatively, caprate (n-decanoate); alternatively, n-undecanoate; alternatively, laurate (n-dodecanoate); or alternatively, stearate (n-octadecanoate).

In an aspect, the chromium compound for the oligomerization catalyst system can comprise, consist essentially of, or consist of, a chromium(II) carboxylate; or alternatively, a chromium(III) carboxylate. Exemplary chromium(II) carboxylates can include, but are not limited to, chromium(II) acetate, chromium(II) propionate, chromium(II) butyrate, chromium(II) isobutyrate, chromium(II) neopentanoate, chromium(II) oxalate, chromium(II) octanoate, chromium(II) (2-ethylhexanoate), chromium(II) laurate, or chromium(II) stearate; or alternatively, chromium(II) acetate, chromium(II) propionate, chromium(II) butyrate, chromium(II) isobutyrate, chromium(II) neopentanoate, chromium(II) octanoate, chromium(II) (2-ethylhexanoate), chromium(II) laurate, or chromium(II) stearate. In an aspect, the chromium compound utilized in the catalyst system can comprise, consist essentially of, or consist of, chromium(III) acetate, chromium(III) propionate, chromium(III) butyrate, chromium(III) isobutyrate, chromium(III) neopentanoate, chromium(III) oxalate, chromium(III) octanoate, chromium(III) 2-ethylhexanoate, chromium(III) 2,2,6,6,-tetramethylheptanedionate, chromium(III) naphthenate, chromium(III) laurate, or chromium(III) stearate; or alternatively, chromium(III) acetate, chromium(III) propionate, chromium(III) butyrate, chromium(III) isobutyrate, chromium(III) neopentanoate, chromium(III) octanoate, chromium(III) 2-ethylhexanoate, chromium(III) 2,2,6,6,-tetramethylheptanedionate, chromium(III) naphthenate, chromium(III) laurate, or chromium(III) stearate. In a further aspect, the chromium compound for the oligomerization catalyst system can comprise, consist essentially of, or consist of, chromium(II) acetate; alternatively, chromium(II) propionate; alternatively, chromium(II) butyrate; alternatively, chromium(II) isobutyrate; alternatively, chromium(II) neopentanoate; alternatively, chromium(II) oxalate; alternatively, chromium(II) octanoate; alternatively, chromium(II) (2-ethylhexanoate); alternatively, chromium(II) laurate; alternatively, chromium(II) stearate; alternatively, chromium(III) acetate; alternatively, chromium(III) propionate; alternatively, chromium(III) butyrate; alternatively, chromium(III) isobutyrate; alternatively, chromium(III) neopentanoate; alternatively, chromium(III) oxalate; alternatively, chromium(III) octanoate; alternatively, chromium(III) 2-ethylhexanoate; alternatively, chromium(III) 2,2,6,6,-tetramethylheptane dionate; alternatively, chromium(III) naphthenate; alternatively, chromium(III) laurate; or alternatively, chromium(III) stearate. In some aspects, the chromium compound for the oligomerization catalyst system can comprise, consist essentially of, or consist of, chromium(II) 2-ethylhexanoate or chromium(III) 2-ethylhexanote; or alternatively, chromium(III) 2-ethylhexanoate.

Referring now to the organoaluminum compound utilized as a component in the catalyst compositions and oligomerization processes disclosed herein, any suitable organoaluminum compound or any organoaluminum compound disclosed herein can be utilized. An organoaluminum can be any compound containing bonds between carbon and aluminum. In an aspect, the organoaluminum compound can comprise, can consist essentially of, or can consist of, an alkylaluminum compound. In an aspect, the alkylaluminum compound can comprise, can consist essentially of, or can consist of, a trialkylaluminum compound, a dialkylaluminum halide compound, an alkylaluminum dihalide compound, a dialkylaluminum hydride compound, an alkylaluminum dihydride compound, a dialkylaluminum hydrocarboxide compound, an alkylaluminum dihydrocarboxide compound, an alkyl aluminum sesquihalide compound, an alkylaluminum sesquihydrocarboxide compound, or any combination thereof. In some aspects, the alkylaluminum compound can comprise, can consist essentially of, or can consist of, a trialkylaluminum, an alkylaluminum halide, or any combination thereof. In other aspects, the alkylaluminum compound can be a trialkylaluminum; or alternatively, an alkylaluminum halide.

In a non-limiting aspect, useful trialkylaluminum compounds can include trimethylaluminum (TMA), triethylaluminum (TEA), tripropylaluminum, tributylaluminum, trihexylaluminum, trioctylaluminum, or mixtures thereof. In some non-limiting aspects, useful trialkylaluminum compounds can include trimethylaluminum, triethylaluminum, tripropylaluminum, tri-n-butylaluminum, tri-isobutylaluminum (TIBA), trihexylaluminum, tri-n-octylaluminum, or mixtures thereof; alternatively, triethylaluminum, tri-n-butylaluminum, tri-isobutylaluminum, trihexylaluminum, tri-n-octylaluminum, or mixtures thereof; alternatively, triethylaluminum, tri-n-butylaluminum, trihexylaluminum, tri-n-octylaluminum, or mixtures thereof. In other non-limiting aspects, useful trialkylaluminum compounds can include trimethylaluminum; alternatively, triethylaluminum; alternatively, tripropylaluminum; alternatively, tri-n-butylaluminum; alternatively, tri-isobutylaluminum; alternatively, trihexylaluminum; or alternatively, tri-n-octylaluminum.

In an aspect, each halide of any alkylaluminum halide disclosed herein can independently be fluoride, chloride, bromide, or iodide; or alternatively, chloride, bromide, or iodide. In an aspect, each halide of any alkylaluminum halide disclosed herein can be fluoride; alternatively, chloride; alternatively, bromide; or alternatively, iodide. In a non-limiting aspect, useful alkylaluminum halides can include diethylaluminum chloride, diethylaluminum bromide, ethylaluminum dichloride, ethylaluminum sesquichloride, and mixtures thereof. In some non-limiting aspects, useful alkylaluminum halides can include diethylaluminum chloride, ethylaluminum dichloride, ethylaluminum sesquichloride, and mixtures thereof. In other non-limiting aspects, useful alkylaluminum halides can include diethylaluminum chloride; alternatively, diethylaluminum bromide; alternatively, ethylaluminum dichloride; or alternatively, ethylaluminum sesquichloride.

In some aspects, the organoaluminum compound can comprise, can consist essentially of, or can consist of, triethylaluminum (TEA), diethylaluminum chloride (DEAC), or any combination thereof. In other aspects, the organoaluminum compound can be triethylaluminum (TEA), or alternatively, diethylaluminum chloride (DEAC).

In an aspect, each alkyl group of an alkylaluminum compound independently can be a C₁ to C₂₀ alkyl group; alternatively, a C₁ to C₁₀ alkyl group; or alternatively, a C₁ to C₆ alkyl group. In an aspect, each alkyl group of an alkylaluminum compound independently can be a methyl group, an ethyl group, a propyl group, a butyl group, a pentyl group, a hexyl group, a heptyl group, or an octyl group; alternatively, a methyl group, an ethyl group, a butyl group, a hexyl group, or an octyl group. In some aspects, each alkyl group of an alkylaluminum compound can be a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

The aromatic hydrocarbon solvent utilized as a component in the first catalyst composition (and in related oligomerization processes) can be any suitable aromatic hydrocarbon solvent comprising xylenes disclosed herein. In an aspect, the aromatic hydrocarbon solvent can comprise any suitable amount of xylenes including xylene isomers ortho-xylene, meta-xylene, para-xylene, or mixtures thereof, or an amount in any range disclosed herein. For example, the aromatic hydrocarbon solvent comprising xylenes can comprise at least 75 wt. % xylenes, and more often, at least 78 wt. %, at least 80 wt. %, at least 83 wt. %, at least 85 wt. %, or at least 90 wt. % xylenes. In addition to xylenes, the aromatic hydrocarbon solvent also can contain a minor amount of ethylbenzene. While not limited thereto, the aromatic hydrocarbon solvent can comprise xylenes and from 5 to 25 wt. %, from 5 to 20 wt. %, from 10 to 25 wt. %, or from 10 to 20 wt. % ethylbenzene. Beneficially, this aromatic hydrocarbon solvent contains a very small amount of benzene, generally no more than 500 ppmw (ppm by weight). In some aspects, the aromatic hydrocarbon solvent comprising xylenes can contain less than or equal to 250 ppmw, less than or equal to 100 ppmw, less than or equal to 50 ppmw, or less than or equal to 25 ppmw of benzene, while in other aspects, the aromatic hydrocarbon solvent comprising xylenes can contain less than or equal to 10 ppmw, less than or equal to 8 ppmw, less than or equal to 5 ppm, or less than or equal to 3 ppmw benzene.

Referring now to the second catalyst composition, the aromatic hydrocarbon solvent utilized as a component in the second catalyst compositions (and in related oligomerization processes) can comprise C₉ substituted benzenes. Representative and non-limiting examples of C₉ substituted benzenes include, but are not limited to, trimethyl benzenes (e.g., 1,2,4-trimethyl benzene, 1,3,5-trimethyl benzene), methyl ethyl benzenes (e.g., 1,2-ethyl toluene, 1,3-ethyl toluene, 1,4-ethyl toluene), and the like, or any mixture thereof. In an aspect, the aromatic hydrocarbon solvent can comprise any suitable amount of C₉ substituted benzenes or an amount in any range disclosed herein. For example, the aromatic hydrocarbon solvent comprising C₉ substituted benzenes can comprise at least 75 wt. % C₉ substituted benzenes, and more often, at least 78 wt. %, at least 80 wt. %, at least 83 wt. %, at least 85 wt. %, or at least 90 wt. % C₉ substituted benzenes. In addition to C₉ substituted benzenes, the aromatic hydrocarbon solvent also can contain a minor amount of C₁₀+ substituted benzenes. While not limited thereto, the aromatic hydrocarbon solvent can comprise C₉ substituted benzenes and from 5 to 25 wt. %, from 5 to 20 wt. %, from 10 to 25 wt. %, or from 10 to 20 wt. % of C₁₀+ substituted benzenes.

Beneficially, this aromatic hydrocarbon solvent, in addition to C₉ substituted benzenes, contains a very small amount of benzene, generally no more than 500 ppmw (ppm by weight). In some aspects, the aromatic hydrocarbon solvent comprising C₉ substituted benzenes can contains less than or equal to 250 ppmw, less than or equal to 100 ppmw, less than or equal to 50 ppmw, or less than or equal to 25 ppmw of benzene, while in other aspects, the aromatic hydrocarbon solvent comprising C₉ substituted benzenes can contain less than or equal to 10 ppmw, less than or equal to 8 ppmw, less than or equal to 5 ppm, or less than or equal to 3 ppmw benzene.

The first catalyst composition and the second catalyst composition can be prepared by contacting or combining the respective components in any order or sequence. Accordingly, the first and second catalyst compositions can be produced by contacting, in any order, the pyrrole compound, the chromium compound, the organoaluminum compound, and the aromatic hydrocarbon solvent (comprising xylenes or C₉ substituted benzenes). In a particular aspect, the catalyst compositions are prepared by first combining the aromatic hydrocarbon solvent (comprising xylenes or C₉ substituted benzenes) and the organoaluminum compound, then combining the pyrrole compound, and then the chromium compound. Alternatively, the catalyst composition can be prepared by first combining the aromatic hydrocarbon solvent and the pyrrole compound, then combining the organoaluminum compound and then the chromium compound. Alternatively, the catalyst composition can be prepared by first combining the aromatic hydrocarbon solvent and the chromium compound, then combining the organoaluminum compound and then the pyrrole compound.

The relative amounts of the components in the catalyst composition are not particularly limited, i.e., the catalyst system can have any suitable molar ratio, for example, of chromium:aluminum (via the chromium compound and the organoaluminum compound components of the catalyst system). Nonetheless, the molar ratio of chromium:aluminum in the catalyst system can be, but is not limited to, a range from 1:1 to 1:150, from 1:1 to 1:100, from 1:3 to 1:50 or from 1.9 to 1.21, and the like. Additionally or alternatively, the catalyst system can have any suitable molar ratio of pyrrole nitrogen:chromium or a molar ratio in any range disclosed herein. The molar ratio of pyrrole nitrogen:chromium in the catalyst system can fall within a range from 1:1 to 4:1, and more often, the molar ratio is from 1.5:1 to 3.7:1, from 1.5:1 to 2.5:1, from 2:1 to 3.7:1, or from 2.5:1 to 3.5:1. Additionally or alternatively, the catalyst system can have any suitable molar ratio of chromium:solvent (aromatic hydrocarbon solvent) or a molar ratio in any range disclosed herein. In one aspect, the molar ratio of chromium:solvent in the catalyst system can be in a range from 1:10 to 1:10,000, and in another aspect, the molar ratio can range from 1:20 to 1:5,000, and in yet another aspect, the molar ratio can range from 1:25 to 1:2,500, and in still another aspect, the molar ratio can range from 1:30 to 1:1,000.

### Oligomerization Processes

The oligomerization catalyst system described herein may be utilized within an oligomerization process or a process to prepare an oligomerization product. A first oligomerization process consistent with aspects of this invention can comprise (or consist essentially of, or consist of) (i) contacting ethylene, an organic reaction medium, optionally hydrogen, and any of the catalyst compositions disclosed herein in an oligomerization reactor, (ii) forming an oligomer product in the oligomerization reactor, the oligomer product comprising hexenes and higher oligomers, and (iii) discharging an effluent stream from the oligomerization reactor, the effluent stream comprising unreacted ethylene and the oligomer product. A second oligomerization process encompassed herein can comprise (or consist essentially of, or consist of) (I) performing any process to produce the catalyst composition disclosed herein, (II) contacting ethylene, an organic reaction medium, optionally hydrogen, and the catalyst composition in an oligomerization reactor, (III) forming an oligomer product in the oligomerization reactor, the oligomer product comprising hexenes and higher oligomers, and (IV) discharging an effluent stream from the oligomerization reactor, the effluent stream comprising unreacted ethylene and the oligomer product.

Referring now to step (i) of the first process and step (II) of the second process, ethylene, the organic reaction medium, and the catalyst composition (and hydrogen, if used) are contacted in an oligomerization reactor. Ethylene, the organic reaction medium, and the catalyst composition (and hydrogen, if used) can be combined in any order or sequence and introduced into the oligomerization reactor separately or in any combination. This invention is not limited by the manner in which the respective feed streams are introduced into the reactor. In an aspect, ethylene can be introduced into the oligomerization reactor separately from the catalyst composition. Additionally or alternatively, the organic reaction medium and the catalyst composition can be combined to form a mixture and then this mixture can be introduced into the oligomerization reactor.

Contacting and/or reacting the ethylene and the catalyst composition in the oligomerization reactor is carried out in the presence of an organic reaction medium, and this organic reaction medium can comprise a saturated aliphatic hydrocarbon, an aromatic hydrocarbon, a linear alpha olefin, or any combination thereof. The saturated aliphatic hydrocarbon can have any suitable number of carbon atoms. Exemplary saturated aliphatic hydrocarbon compounds include, but are not limited to, propane, butane, pentane, hexane, heptane, octane, cyclohexane, methyl cyclohexane, and the like, as well as any mixture or combination thereof. Aromatic hydrocarbons that may be used as the organic reaction medium can include, but are not limited to, C₆ to C₅₀ aromatic compounds; alternatively, C₆ to C₃₀ aromatic compounds; alternatively, C₆ to C₁₈ aromatic compounds; or alternatively, C₆ to C₁₀ aromatic compounds. Exemplary aromatic hydrocarbons include, but are not limited to, benzene, toluene, xylenes, cumene, ethylbenzene, C₉ substituted benzenes (e.g., 1,2,4-trimethyl benzene, 1,3,5-trimethyl benzene, 1,2-ethyl toluene, 1,3-ethyl toluene, 1,4-ethyl toluene), and the like, as well as any mixture or combination thereof. Linear alpha olefins that can be used as the organic reaction medium can include, but are not limited to, 1-butene, 1-hexene, 1-octene, 1-decene, 1-dodecene, 1-tetradecene, and the like, as well as any mixture or combination thereof.

Referring now to step (ii) of the first process and step (III) of the second process, an oligomer product is formed in the oligomerization reactor, and the oligomer product comprises hexenes and higher oligomers. The oligomerization reactor in which the oligomer product is formed in step (ii) and step (III) (and in which the components in step (i) and step (II) are contacted) can comprise any suitable reactor. Non-limiting examples of reactor types can include a stirred tank reactor, a plug flow reactor, or any combination thereof; alternatively, a fixed bed reactor, a continuous stirred tank reactor, a loop reactor, a solution reactor, a tubular reactor, a recycle reactor, or any combination thereof. For instance, in one aspect, the oligomerization reactor can comprise a continuous stirred tank reactor, while in another aspect, the oligomerization reactor can comprise a loop reactor. In some aspects, there can be more than one reactor in series or in parallel, and including any combination of reactor types and arrangements. Moreover, the oligomerization process used to form the oligomer product can be a continuous process or a batch process, or any reactor or vessel utilized in the process can be operated continuously or batchwise.

Forming the oligomer product in the oligomerization reactor can be accomplished at any suitable oligomerization temperature and pressure. Often, the oligomer product can be formed at a minimum temperature of 0° C., 20° C., 30° C., 40° C., 45° C., or 50° C.; additionally or alternatively, at a maximum temperature of 165° C., 160° C., 150° C., 140° C., 130° C., 115° C., 100° C., or 90° C. Generally, the oligomerization temperature at which the oligomer product is formed can be in a range from any minimum temperature disclosed herein to any maximum temperature disclosed herein. Accordingly, suitable non-limiting ranges can include the following: from 0 to 165, from 20 to 160, from 20 to 115, from 40 to 160, from 40 to 140, from 50 to 150, from 50 to 140, from 50 to 130, from 50 to 100, from 60 to 115, from 70 to 100, or from 75 to 95° C. Other appropriate oligomerization temperatures and temperature ranges are readily apparent from this disclosure.

The oligomer product can be formed at a minimum pressure (or ethylene partial pressure) of 50 psig (344 kPa), 100 psig (689 kPa), 200 psig (1.4 MPa), or 250 psig (1.5 MPa); additionally or alternatively, at a maximum pressure (or ethylene partial pressure) of 4,000 psig (27.6 MPa), 3,000 psig (20.9 MPa), 2,000 psig (13.8 MPa), or 1,500 psig (10.3 MPa). Generally, the oligomerization pressure (or ethylene partial pressure) at which the oligomer product is formed can be in a range from any minimum pressure disclosed herein to any maximum pressure disclosed herein. Accordingly, suitable non-limiting ranges can include the following: from 50 psig (344 kPa) to 4,000 psig (27.6 MPa), from 100 psig (689 kPa) to 3,000 psig (20.9 MPa), from 100 psig (689 kPa) to 2,000 psig (13.8 MPa), from 200 psig (1.4 MPa) to 2,000 psig (13.8 MPa), from 200 psig (1.4 MPa) to 1,500 psig (10.3 MPa), or from 250 psig (1.5 MPa) to 1,500 psig (10.3 MPa). Other appropriate oligomerization pressures (or ethylene partial pressures) are readily apparent from this disclosure.

Optionally, hydrogen can be added to the reactor either directly or hydrogen can be combined with an ethylene feed prior to the reactor. In the reactor, the hydrogen partial pressure can be at least 1 psig (6.9 kPa), 5 psig (34 kPa), 10 psig (69 kPa), 25 psig (172 kPa), or 50 psig (345 kPa); additionally or alternatively, a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), from 25 psig (172 kPa) to 400 psig (2.8 MPa), or from 50 psig (345 kPa) to 500 psig (3.4 MPa). Other appropriate hydrogen partial pressures in the reactor for the formation of the oligomer product are readily apparent from this disclosure.

In step (iii) of the first process and step (IV) of the second process, an effluent stream is discharged from the oligomerization reactor, and the effluent stream contains unreacted ethylene and the oligomer product. The amount of conversion of ethylene in the oligomerization reactor is not particularly limited, and generally the minimum ethylene conversion can be at least at least 40, 50, 60, or 65 wt. %, while the maximum ethylene conversion can be 99, 95, 90, 80, 75, or 70 wt. %. Generally, the ethylene conversion in the reactor can range from any minimum conversion to any maximum conversion described herein. For instance, the ethylene conversion can range from 40 to 99 wt. %, from 50 to 95 wt. %, from 60 to 90 wt. %, from 60 to 80 wt. %, from 65 to 80 wt. %, from 65 to 75 wt. %, or from 65 to 70 wt. %. The ethylene conversion is based on the amount of ethylene entering the reactor and the amount of (unreacted) ethylene in the effluent stream.

Among other constituents, the effluent stream contains the oligomer product, which can comprise hexenes. The amount of hexenes in the oligomer product typically can fall within a range from 50 to 99 wt. % based on the total amount of oligomers in the oligomer product. In an aspect, the minimum amount of hexenes in the oligomer product can be at least 50, 60, 70, 80, 85, or 90 wt, %. In another aspect, the maximum amount of hexenes in the oligomer product can be 99, 98, 95, or 93 wt. %. Generally, the amount of hexenes in the oligomer product can range from any minimum amount of hexenes in the oligomer product to any maximum amount of hexenes in the oligomer product described herein. For instance, the amount of hexenes—based on the total weight of oligomers in the oligomer product—can be from 50 to 99 wt. %, from 60 to 93 wt. %, from 70 to 98 wt. %, from 80 to 98 wt. %, from 85 to 95 wt. %, or from 90 to 95 wt. % hexenes.

Optionally, the disclosed oligomerization processes can further comprise a step of contacting the effluent stream—after discharging from the oligomerization reactor—with a catalyst system deactivating agent. While not limited thereto, the catalyst system deactivating agent can be a C₄-C₁₈ alcohol, and the term alcohol is used generically to include mono-ols, diols, and polyols, therefore the catalyst system deactivating agent can comprise a mono alcohol compound, a diol compound, a polyol compound, or any combination thereof.

Consistent with particular aspects of this invention, the catalyst system deactivating agent can comprise a butanol, a pentanol, a hexanol, a heptanol, an octanol, a nonanol, a decanol, an undecanol, and the like, as well as any mixture or combination thereof. Specific examples of catalyst system deactivating agents that can be utilized herein include, for instance, 1-butanol, 2-butanol, iso-butanol, sec-butanol, t-butanol, 1-pentanol, cyclopentanol, 1-hexanol, 2-hexanol, 3-hexanol, cyclohexanol, 1-heptanol, 2-heptanol, 3-heptanol, 4-heptanol, 1-octanol, 2-octanol, 3-octanol, 4-octanol, 2-ethyl-1-hexanol, 2-methyl-3-heptanol, 1-nonanol, 1-decanol, 2-decanol, 3-decanol, 4-decanol, 5-decanol, 1-undecanol, 2-undecanol, 7-methyl-2-decanol, a 1-docecanol, a 2-dodecanol, 2-ethyl-1-decanol, and the like, as well any mixture or combination thereof. In a particular aspect disclosed herein, the catalyst system deactivating agent can comprise 2-ethyl-hexanol.

Optionally, the disclosed oligomerization processes—after discharging the effluent stream from the reactor—can further comprise a step of separating at least a portion of the unreacted ethylene from the effluent stream. This unreacted ethylene is typically recycled. While not limited thereto, a typical technique used to separate unreacted ethylene from the effluent stream is flashing via pressure reduction, such as from a reactor pressure of over 500 psig to a pressure of less than 200 psig (or less than 150 psig).

Optionally, the disclosed oligomerization processes can further comprise a step of separating (or isolating) a C₆ stream from the oligomer product. The separated (or isolated) C₆ stream can contain any suitable amount of hexenes, such as at least 96, at least 97, at least 98, at least 99 wt. %, or at least 99.5 wt. % hexenes, based on the total weight of the C₆ stream. Any suitable fractionation scheme can be employed to separate (or isolate) the C₆ stream from the oligomer product, such as the use of one, two, or three (or more) distillation columns.

Generally, a vast majority of the C₆ stream is the desirable α-olefin product, 1-hexene. While not limited thereto, the C₆ stream can contain, for example, at least 90 wt. % 1-hexene, and more often, at least 95 wt. %, at least 97 wt. %, at least 98 wt. %, at least 99 wt. %, or at least 99.5 wt. % 1-hexene, based on the total weight of the hexenes in the C₆ stream.

Beneficially, this C₆ stream, in addition to 1-hexene, contains a very small amount of benzene, generally no more than 10 ppmw (ppm by weight). In some aspects, the C₆ stream can contain less than or equal to 5 ppmw, less than or equal to 2 ppmw, less than or equal to 1 ppmw, or less than or equal to 0.5 ppmw of benzene, while in other aspects, the C₆ stream can contain 1-hexene and less than or equal to 0.2 ppmw, less than or equal to 0.15 ppmw, less than or equal to 0.1 ppmw, or less than 0.05 ppmw of benzene.

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

The oligomerization studies of Examples 1-8 were carried out to compare the catalytic behavior of catalyst systems utilizing various aromatic hydrocarbon solvents summarized in Table 1, under standard selective 1-hexene oligomerization reaction conditions. Additionally, these studies were conducted to identify aromatic hydrocarbon solvents (that contain less than 100 ppm of benzene) for active ethylene trimerization catalyst systems that are selective towards C₆ products, specifically 1-hexene, while minimizing undesirable oligomer byproducts. For instance, a high purity 1-hexene grade may require the presence of no more than 0.2 ppm of benzene, which in turn can translate to no more than 25 ppm (or no more than 10 ppm) of benzene present in the aromatic hydrocarbon solvent used in catalyst preparation.

### Examples 1-8

The catalyst compositions utilized in Examples 1-8 were prepared as follows. A pyrrole compound, 2,5-dimethylpyrrole (0.13 g, 1.4 mmol), was added to diethylaluminum chloride (0.46 g, 3.8 mmol) and triethylaluminum (0.65 g, 5.7 mmol) in 3.42 g of the respective aromatic hydrocarbon solvent of Examples 1-8, the compositions of which are summarized in Table 1. Chromium (II) ethylhexanoate (7.25 wt. % Cr in 1-dodecene) (0.34 g, 0.47 mmol) was then added and stirred for 90 min.

Ethylene oligomerization experiments of Examples 1-8 were performed as follows. Dry cyclohexane (480 mL, 4.41 mol) was mixed with a sample of the activated catalyst system (0.264 g for 3.4 ppm Cr runs or 0.115 g for 1.5 ppm Cr runs). The diluted activated catalyst system was then charged into a Swagelok block valve-adapted stainless-steel cylinder. This catalyst charge was then nitrogen-flushed into a one-liter Autoclave Engineers Zipperclave® reactor, pre-heated to 65° C. After hydrogen equilibration at 50 psig and heating to 75° C., 750 psig of ethylene was added into the reactor and the reaction temperature was set to 110° C. At reaction completion (30 min), the reactor was cooled to ambient temperatures before the unreacted ethylene and hydrogen gas were vented from the reactor. A sample of the reactor contents was then collected, isolated, and analyzed by GC. Each example was tested at least three times to establish a standard deviation. Gas chromatographic (GC) analyses were performed using a split injection method on an Agilent Technologies 7890B gas chromatograph with a flame ionization detector (FID). Initial oven temperature was 50° C. for 10 min, increased at 8° C./min to 260° C. with a 15 min hold time. The column was a polysiloxane capillary column (Agilent J&W™ DB-1, 60 m×0.320 mm×1 micron film thickness). Productivity and distribution data were calculated from product ratios and referenced against a known internal standard. Data analysis was performed using Agilent Chemstation software.

Table 1 summarizes the GC compositional breakdown of the aromatic hydrocarbon solvents of Examples 1-8 that were utilized in the formation of catalyst compositions. Comparative Example 1 used an ethylbenzene solvent, Examples 2-6 used xylene solvents, and Examples 7-8 used C₉ substituted benzene solvents. The percentages in Table 1 were determined in area percentages via gas chromatography, but the values are substantially the same as weight percentages. Meta-xylene and para-xylene exhibit similar retention times via gas chromatography, so a ratio of the combination of the two components relative to ortho-xylene is reported in Table 1. Gas chromatographic (GC) analyses were performed using a split injection method on an Agilent Technologies 7890B gas chromatograph with a flame ionization detector (FID). Initial oven temperature was 35° C. for 13 min, increased at 10° C./min to 45° C. with a 15 min hold time, then increased at 1° C./min to 60° C. with a 15 min hold time, followed by an increase of 1.9° C./min to 250° C. with a 30 min hold time. The column was a fused silica capillary column (Supelco Petrocol™ DH, 100 m×0.25 mm×0.5 micron film thickness). Data analysis was performed using Agilent Chemstation software.

As shown in Table 1, the low benzene content of the aromatic hydrocarbon solvents of Examples 2-8 (from 0.3 to 60 ppm) compared to the high benzene content of the ethylbenzene solvent of Comparative Example 1 (403 ppm) suggests that the residual benzene content in 1-hexene products produced in the ethylene oligomerization process could be reduced significantly. Beneficially, by utilizing an aromatic hydrocarbon with a low benzene content (e.g., less than 25 ppm or less than 10 ppm) in catalyst system preparation, pre-distillation of the aromatic solvent (to remove benzene) can be eliminated, thus simplifying catalyst preparation and the oligomerization process, and reducing cost and waste.

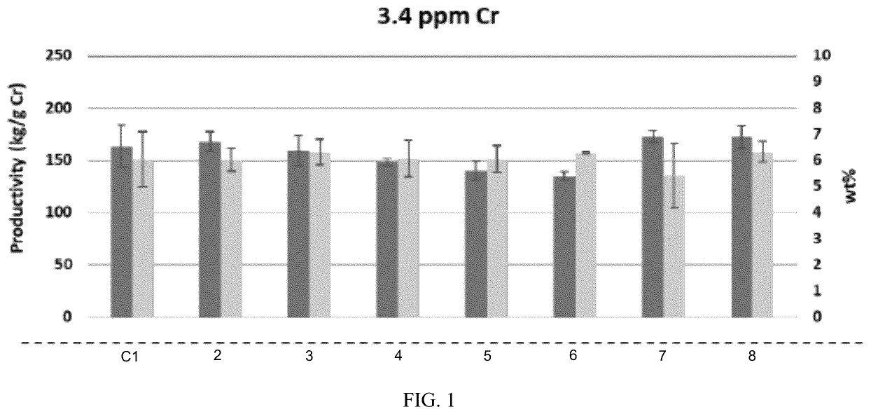

For Examples 1-8, FIG. 1 illustrates the 1-hexene productivity (left axis, first bar for each example) and C₁₀ wt. % (right axis, second bar for each example) at a 3.4 ppm reactor concentration of chromium. The productivity and C₁₀ amount for each of Examples 2-8 were generally statistically the same as that of Comparative Example 1. Example 6 had the lowest 1-hexene productivity. Productivity and distribution data were calculated from product ratios which were referenced against the known ethylbenzene component as an internal standard. The ethylbenzene weight percentage in the xylene solvent mixture was independently measured to ensure accurate referencing of product ratios. Product distributions from the heavier aromatic solvent (≥C₉ substituted benzenes) mixture evaluation runs which did not contain any inherent ethylbenzene were referenced to an added nonane internal standard.

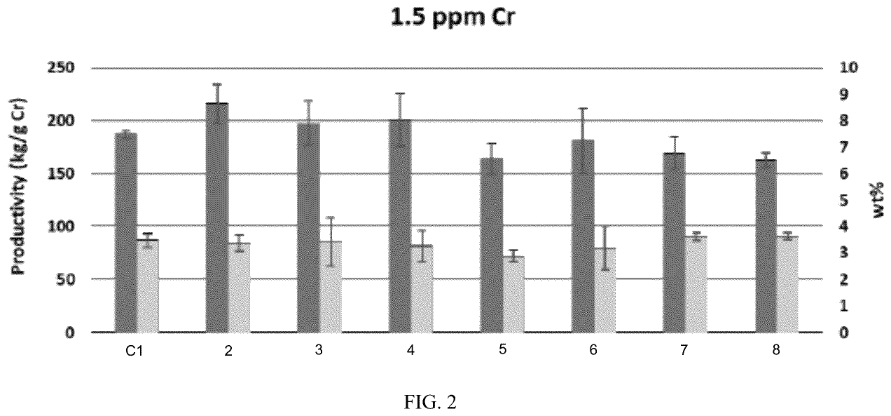

FIG. 2 is similar to FIG. 1, but at the lower reactor concentration of chromium (1.5 ppm). Given the standard deviation across the experiments, all examples performed roughly the same in productivity, although Example 2 did show a marginal improvement in 1-hexene productivity over Comparative Example 1. Lower C₁₀ byproduct weight percentages in FIG. 2 were observed for all examples at the lower chromium concentration, as compared to FIG. 1.

Examples 1-8 had the same 1-hexene purity. The 1-hexene product purity at 3.4 ppm Cr averaged ˜99.4% 1-hexene (based on all C₆ products) for each example. However, at the lower 1.5 ppm reactor concentration, the average was a slightly higher ˜99.6% 1-hexene purity with very little deviation. In sum, the aromatic hydrocarbon solvents of Examples 2-7 performed similarly to that of Comparative Example 1 in overall ethylene oligomerization performance.

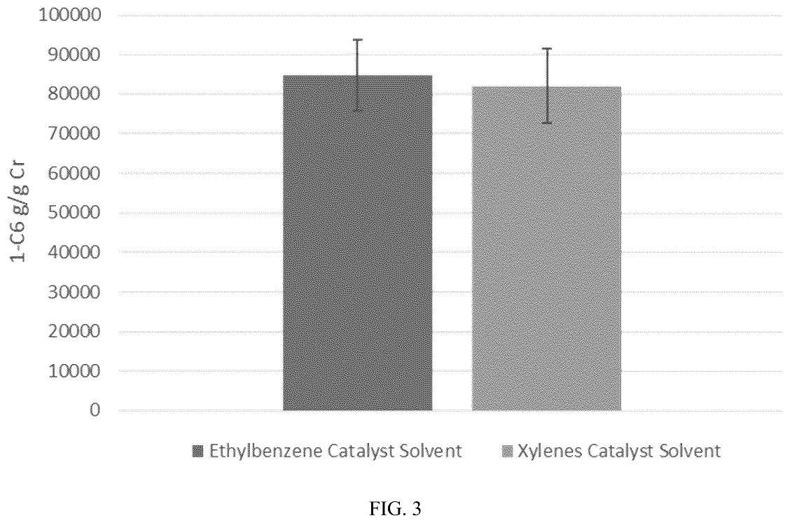

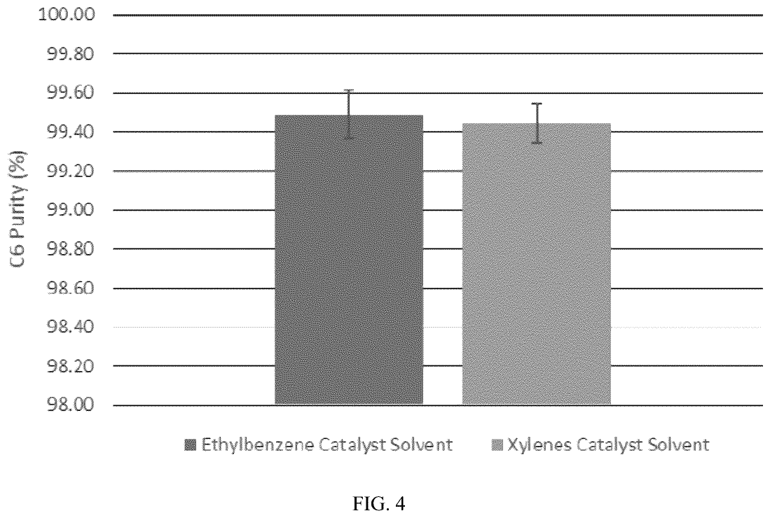

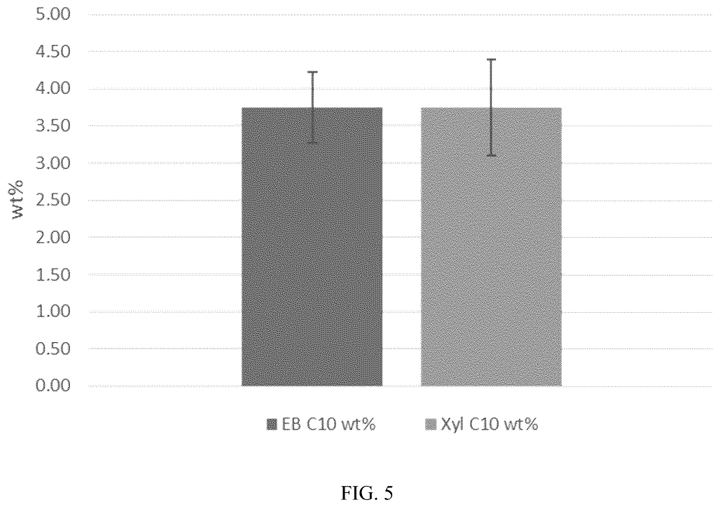

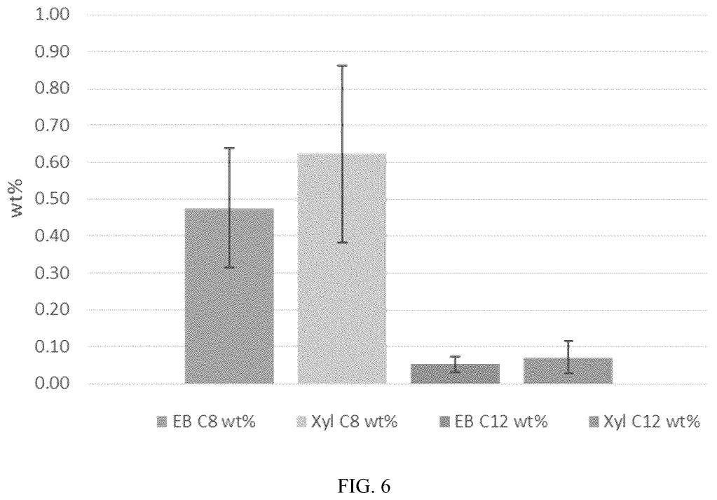

FIGS. 3-6 compare the performance of Comparative Example 1 (ethylbenzene) and Example 2 (xylenes) at the 3.4 ppm chromium concentration for C₆ productivity (g 1-C6/g Cr), C₆ product purity (wt. %), C₁₀ byproduct (wt. %), and C₈ and C₁₂ byproducts (wt. %), respectively. The 1-hexene productivity in FIG. 3 was statistically the same for catalysts prepared in ethylbenzene and for catalysts prepared in xylenes. Hexene purity in FIG. 4 was not affected by solvent selection, averages of 99.48% and 99.44% purity were observed for catalysts prepared in ethylbenzene versus xylenes, respectively. C₁₀ byproduct weight percent in FIG. 5 was the same regardless of the solvent type. FIG. 6 shows slightly higher C₈ and C₁₂ byproducts when using the xylene solvent of Example 2, but the differences appear to be statistically insignificant.

TABLE 1

| | | Benzene | | | | C₉ | >C₉ |
|---|---|---|---|---|---|---|---|
| | | content | Ethylbenzene | Xylenes | (m/p-Xylene)/ | Benzenes | Benzenes |
| Example | Main Solvent | (ppm) | (wt. %) | (wt. %) | (o-Xylene) | (wt. %) | (wt. %) |
| C1 | Ethylbenzene | 403.0 | 99.41 | 0.4 | 4.96 | <0.1 | <0.1 |
| 2 | Xylenes | 17.5 | 15.97 | 83.73 | 4.27 | 0.15 | <0.01 |
| 3 | Xylenes | 8.6 | 12.62 | 86.73 | 2.99 | 0.53 | 0.01 |
| 4 | Xylenes | 22.0 | 12.71 | 86.88 | 2.64 | 0.33 | <0.01 |
| 5 | Xylenes | 57.7 | 13.38 | 85.62 | 2.89 | 0.78 | 0.01 |
| 6 | Xylenes | 2.5 | 18.34 | 81.52 | 5.34 | 0.14 | <0.01 |
| 7 | C₉ substituted | 0.3 | 0.02 | 0.67 | 0.19 | 84.75 | 14.57 |
| | benzenes | | | | | | |
| 8 | C₉ substituted | 11.5 | 0.01 | 0.92 | 0.14 | 84.05 | 14.97 |
| | benzenes | | | | | | |

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A catalyst composition comprising (a) a pyrrole compound, (b) a chromium compound, (c) an organoaluminum compound, and (d) an aromatic hydrocarbon solvent comprising xylenes.

Aspect 2. The composition defined in aspect 1, wherein the aromatic hydrocarbon solvent comprises any suitable amount of xylenes or an amount in any range disclosed herein, e.g., at least 75 wt. %, at least 78 wt. %, at least 80 wt. %, at least 83 wt. %, at least 85 wt. %, or at least 90 wt. % xylenes.

Aspect 3. The composition defined in aspect 1 or 2, wherein the aromatic hydrocarbon solvent comprises any suitable amount of ethylbenzene or an amount in any range disclosed herein, e.g., from 5 to 25 wt. %, from 5 to 20 wt. %, from 10 to 25 wt. %, or from 10 to 20 wt. % ethylbenzene.

Aspect 4. A process to produce the catalyst composition defined in any one of aspects 1-3, the process comprising contacting, in any order, (a) the pyrrole compound, (b) the chromium compound, (c) the organoaluminum compound, and (d) the aromatic hydrocarbon solvent.

Aspect 5. The process defined in aspect 4, wherein the process comprises first combining the aromatic hydrocarbon solvent and the organoaluminum compound, then combining the pyrrole compound, and then the chromium compound.

Aspect 6. A catalyst composition comprising (A) a pyrrole compound, (B) a chromium compound, (C) an organoaluminum compound, and (D) an aromatic hydrocarbon solvent comprising C₉ substituted benzenes.

Aspect 7. The composition defined in aspect 6, wherein the aromatic hydrocarbon solvent comprises any suitable amount of C₉ substituted benzenes or an amount in any range disclosed herein, e.g., at least 75 wt. %, at least 78 wt. %, at least 80 wt. %, at least 83 wt. %, at least 85 wt. %, or at least 90 wt. % C₉ substituted benzenes.

Aspect 8. The composition defined in aspect 6 or 7, wherein the aromatic hydrocarbon solvent comprises any suitable amount of C₁₀+ substituted benzenes or an amount in any range disclosed herein, e.g., from 5 to 25 wt. %, from 5 to 20 wt. %, from 10 to 25 wt. %, or from 10 to 20 wt. % C₁₀+ substituted benzenes.

Aspect 9. The composition defined in any one of aspects 6-8, wherein the C₉ substituted benzenes comprise any suitable C₉ substituted benzenes or any disclosed herein, e.g., a trimethyl benzene, a methyl ethyl benzene, or any combination thereof.

Aspect 10. A process to produce the catalyst composition defined in any one of aspects 6-9, the process comprising contacting, in any order, (A) the pyrrole compound, (B) the chromium compound, (C) the organoaluminum compound, and (D) the aromatic hydrocarbon solvent.

Aspect 11. The process defined in aspect 10, wherein the process comprises first combining the aromatic hydrocarbon solvent and the organoaluminum compound, then combining the pyrrole compound, and then the chromium compound.

Aspect 12. The composition or process defined in any one of aspects 1-11, wherein the aromatic hydrocarbon solvent comprises any suitable amount of benzene or an amount in any range disclosed herein, e.g., less than or equal to 500 ppmw (ppm by weight), less than or equal to 250 ppmw, less than or equal to 100 ppmw, less than or equal to 50 ppmw, less than or equal to 25 ppmw, less than or equal to 10 ppmw, less than or equal to 8 ppmw, less than or equal to 5 ppm, or less than or equal to 3 ppmw benzene.

Aspect 13. The composition or process defined in any one of aspects 1-12, wherein the pyrrole compound comprises any suitable pyrrole compound or any pyrrole compound disclosed herein, e.g., having formula (A), wherein R, R¹, R², and R³ independently are H or a C₁ to C₃₆ hydrocarbyl group:

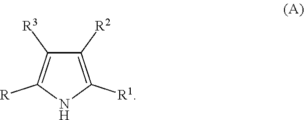

Aspect 14. The composition or process defined in any one of aspects 1-13, wherein the pyrrole compound comprises 2,5-dimethylpyrrole, 2,5-diethylpyrrole, or both.

Aspect 15. The composition or process defined in any one of aspects 1-14, wherein the chromium compound comprises any suitable chromium compound or any chromium compound disclosed herein, e.g., a chromium (II) or chromium (III) nitrate, sulfate, halide, alkoxide, carboxylate, or beta-dionate (or acetylacetonate), or any combination thereof.

Aspect 16. The composition or process defined in any one of aspects 1-15, wherein the chromium compound comprises chromium (III) 2-ethylhexanoate, chromium (III) octanoate, chromium (III) naphthenate, chromium (III) acetate, chromium (III) propionate, chromium (III) butyrate, chromium (III) neopentanoate, chromium (III) laurate, chromium (III) stearate, chromium (III) oxalate, chromium (II) bis(2-ethylhexanoate), chromium (II) acetate, chromium (II) propionate, chromium (II) butyrate, chromium (II) neopentanoate, chromium (II) laurate, chromium (II) stearate, chromium (II) oxalate, or any combination thereof.

Aspect 17. The composition or process defined in any one of aspects 1-16, wherein the chromium compound comprises chromium (III) 2-ethylhexanoate.

Aspect 18. The composition or process defined in any one of aspects 1-17, wherein the organoaluminum compound comprises any suitable organoaluminum compound or any organoaluminum compound disclosed herein, e.g., a trialkylaluminum compound, a dialkylaluminum halide compound, an alkylaluminum dihalide compound, a dialkylaluminum hydride compound, an alkylaluminum dihydride compound, a dialkylaluminum hydrocarboxide compound, an alkylaluminum dihydrocarboxide compound, an alkyl aluminum sesquihalide compound, an alkylaluminum sesquihydrocarboxide compound, or any combination thereof.

Aspect 19. The composition or process defined in any one of aspects 1-18, wherein the organoaluminum compound comprises trimethylaluminum (TMA), triethylaluminum (TEA), tripropylaluminum, tri-n-butylaluminum, triisobutylaluminum (TIBA), diethylaluminum chloride (DEAC), diethylaluminum bromide, ethylaluminum dichloride, ethylaluminum sesquichloride, diethylaluminum ethoxide, or any combination thereof.

Aspect 20. The composition or process defined in any one of aspects 1-19, wherein the organoaluminum compound comprises triethylaluminum (TEA), diethylaluminum chloride (DEAC), or a combination thereof.

Aspect 21. An oligomerization process comprising (i) contacting ethylene, an organic reaction medium, optionally hydrogen, and the catalyst composition defined in any one of aspects 1-3, 6-9, or 12-20 in an oligomerization reactor, (ii) forming an oligomer product in the oligomerization reactor, the oligomer product comprising hexenes and higher oligomers, and (iii) discharging an effluent stream from the oligomerization reactor, the effluent stream comprising unreacted ethylene and the oligomer product.

Aspect 22. An oligomerization process comprising (I) performing the process to produce the catalyst composition defined in any one of aspects 4-5 or 10-20, (II) contacting ethylene, an organic reaction medium, optionally hydrogen, and the catalyst composition in an oligomerization reactor, (III) forming an oligomer product in the oligomerization reactor, the oligomer product comprising hexenes and higher oligomers, and (IV) discharging an effluent stream from the oligomerization reactor, the effluent stream comprising unreacted ethylene and the oligomer product.

Aspect 23. The process defined in aspect 21 or 22, wherein the organic reaction medium comprises a saturated aliphatic hydrocarbon, an aromatic hydrocarbon, a linear α-olefin, or any combination thereof.

Aspect 24. The process defined in aspect 23, wherein the saturated aliphatic hydrocarbon comprises propane, butane, pentane, hexane, heptane, octane, cyclohexane, methyl cyclohexane, or combinations thereof.

Aspect 25. The process defined in aspect 23, wherein the aromatic hydrocarbon comprises benzene, toluene, xylenes, cumene, ethylbenzene, or combinations thereof.

Aspect 26. The process defined in aspect 23, wherein the linear α-olefin comprises 1-butene, 1-hexene, 1-octene, 1-decene, 1-dodecene, 1-tetradecene, or combinations thereof.

Aspect 27. The process defined in any one of aspects 21-26, wherein the oligomerization reactor comprises any suitable reactor type, e.g., a continuous stirred tank reactor, a loop reactor, or any combination thereof.

Aspect 28. The process defined in any one of aspects 21-27, wherein ethylene is introduced into the oligomerization reactor separately from the catalyst composition.

Aspect 29. The process defined in any one of aspects 21-28, wherein the organic reaction medium and the catalyst composition are combined and then introduced into the oligomerization reactor.

Aspect 30. The process defined in any one of aspects 21-29, wherein the oligomer product is formed at any suitable oligomerization temperature and oligomerization pressure or any oligomerization temperature and pressure disclosed herein.

Aspect 31. The process defined in any one of aspects 21-30, wherein the oligomer product comprises any amount of hexenes disclosed herein, e.g., at least 50, 60, 70, 80, 85, or 90 wt. %; a maximum of 99, 98, 95, or 93 wt. %; or from 50 to 99 wt. %, from 60 to 93 wt. %, from 70 to 98 wt. %, from 80 to 98 wt. %, from 85 to 95 wt. %, or from 90 to 95 wt. % hexenes, based on the total amount of oligomers in the oligomer product.

Aspect 32. The process defined in any one of aspects 21-31, wherein the oligomerization reactor has any ethylene conversion disclosed herein, e.g., at least 40, 50, 60, or 65 wt. %; a maximum of 99, 95, 90, 80, 75, or 70 wt. %; or from 40 to 99 wt. %, from 50 to 95 wt. %, from 60 to 90 wt. %, from 60 to 80 wt. %, from 65 to 80 wt. %, from 65 to 75 wt. %, or from 65 to 70 wt. % conversion, based on the amount of ethylene entering the reactor and the amount of ethylene in the effluent stream.

Aspect 33. The process defined in any one of aspects 21-32, further comprising a step of contacting the effluent stream with a catalyst system deactivating agent.

Aspect 34. The process defined in any one of aspects 21-33, further comprising a step of separating at least a portion of the unreacted ethylene from the effluent stream.

Aspect 35. The process defined in any one of aspects 21-34, further comprising a step of separating/isolating a C₆ stream comprising any amount of hexenes disclosed herein, e.g., at least 96, 97, 98, 99, or 99.5 wt. % hexenes, from the oligomer product.

Aspect 36. The process defined in aspect 35, wherein the C₆ stream comprises any amount of 1-hexene disclosed herein, e.g., at least 90, 95, 97, 98, 99, or 99.5 wt. % 1-hexene, based on the total weight of the hexenes.

Aspect 37. The process defined in aspect 35 or 36, wherein the C₆ stream comprises any suitable amount of benzene or an amount in any range disclosed herein, e.g., less than or equal to 10 ppmw (ppm by weight), less than or equal to 5 ppmw, less than or equal to 2 ppmw, less than or equal to 1 ppmw, less than or equal to 0.5 ppmw, less than or equal to 0.2 ppmw, less than or equal to 0.15 ppmw, less than or equal to 0.1 ppmw, or less than or equal to 0.05 ppmw benzene.

Aspect 38. The process defined in any one of aspects 21-37, wherein hydrogen is present in step (i) and/or hydrogen is present in step (II).

Aspect 39. The composition or process defined in any one of aspects 1-38, wherein the catalyst composition has any suitable molar ratio of chromium:aluminum or a molar ratio in any range disclosed herein, e.g., from 1:1 to 1:150, from 1:1 to 1:100, from 1:3 to 1:50, or from 1:9 to 1:21.

Aspect 40. The composition or process defined in any one of aspects 1-39, wherein the catalyst composition has any suitable molar ratio of pyrrole nitrogen:chromium or a molar ratio in any range disclosed herein, e.g., from 1:1 to 4:1, from 1.5:1 to 3.7:1; from 1.5:1 to 2.5:1, from 2:1 to 3.7:1, or from 2.5:1 to 3.5:1.

Aspect 41. The composition or process defined in any one of aspects 1-40, wherein the catalyst composition has any suitable molar ratio of chromium:solvent or a molar ratio in any range disclosed herein, e.g., from 1:10 to 1:10,000, from 1:20 to 1:5,000, from 1:25 to 1:2,500, or from 1:30 to 1:1,000.

What is claimed is:

1. An oligomerization process comprising:
(i) contacting ethylene, an organic reaction medium, optionally hydrogen, and a catalyst composition in an oligomerization reactor, wherein the catalyst composition comprises:
(a) a pyrrole compound;
(b) a chromium compound;
(c) an organoaluminum compound; and
(d) an aromatic hydrocarbon solvent comprising xylenes;
(ii) forming an oligomer product in the oligomerization reactor, the oligomer product comprising hexenes and higher oligomers; and
(iii) discharging an effluent stream from the oligomerization reactor, the effluent stream comprising unreacted ethylene and the oligomer product.

2. The process of claim 1, wherein the aromatic hydrocarbon solvent comprises:
at least 75 wt. % xylenes; and/or
from 5 to 25 wt. % ethylbenzene.

3. The process of claim 1, wherein the aromatic hydrocarbon solvent comprises less than or equal to 250 ppmw (ppm by weight) benzene.

4. The process of claim 1, wherein the aromatic hydrocarbon solvent comprises less than or equal to 25 ppmw (ppm by weight) benzene.

5. The process of claim 1, wherein the catalyst composition has:
a molar ratio of chromium:aluminum from 1:1 to 1:150;
a molar ratio of pyrrole nitrogen:chromium from 1:1 to 4:1; and
a molar ratio of chromium:solvent from 1:10 to 1:10,000.

6. The process of claim 1, wherein the catalyst composition has:
a molar ratio of chromium:aluminum from 1:9 to 1:21;
a molar ratio of pyrrole nitrogen:chromium from 2.5:1 to 3.5:1; and
a molar ratio of chromium:solvent from 1:300 to 1:1,000.

7. The process of claim 1, wherein:
the pyrrole compound comprises 2,5-dimethylpyrrole, 2,5-diethylpyrrole, or both;
the chromium compound comprises chromium (III) 2-ethylhexanoate; and
the organoaluminum compound comprises triethylaluminum (TEA), diethylaluminum chloride (DEAC), or a combination thereof.

8. The process of claim 1, wherein hydrogen is present in step (i).

9. The process of claim 1, wherein the oligomer product comprises from 50 to 99 wt. % hexenes, based on a total amount of oligomers in the oligomer product.

10. The process of claim 1, wherein the oligomerization reactor has an ethylene conversion from 40 to 99 wt. %, based on an amount of ethylene entering the reactor and an amount of unreacted ethylene in the effluent stream.

11. The process of claim 1, further comprising a step of contacting the effluent stream with a catalyst system deactivating agent.

12. The process of claim 1, further comprising a step of separating at least a portion of the unreacted ethylene from the effluent stream.

13. The process of claim 1, further comprising a step of separating or isolating a C₆ stream comprising at least 96 wt. % hexenes from the oligomer product.

14. The process of claim 13, wherein the C₆ stream comprises less than or equal to 5 ppmw (ppm by weight) benzene.

15. The process of claim 13, wherein the C₆ stream comprises at least 90 wt. % 1-hexene, based on a total weight of the hexenes.

16. The process of claim 15, wherein the C₆ stream comprises less than or equal to 0.1 ppmw (ppm by weight) benzene.

17. The process of claim 1, wherein:
ethylene is introduced into the oligomerization reactor separately from the catalyst composition; and/or
the organic reaction medium and the catalyst composition are combined and then introduced into the oligomerization reactor.

18. The process of claim 1, wherein:
the oligomerization reactor comprises a continuous stirred tank reactor, a loop reactor, or any combination thereof; and
the oligomer product is formed at an oligomerization temperature of from 50 to 130° C. and an oligomerization pressure of from 250 psig (1.5 MPa) to 1,500 psig (10.3 MPa).

19. An oligomerization process comprising:
(I) combining an aromatic hydrocarbon solvent and an organoaluminum compound, then combining a pyrrole compound, and then a chromium compound, to form a catalyst composition;
(II) contacting ethylene, an organic reaction medium, optionally hydrogen, and the catalyst composition in an oligomerization reactor;
(III) forming an oligomer product in the oligomerization reactor, the oligomer product comprising hexenes and higher oligomers; and
(IV) discharging an effluent stream from the oligomerization reactor, the effluent stream comprising unreacted ethylene and the oligomer product.

20. A catalyst composition comprising:
(A) a pyrrole compound;
(B) a chromium compound;
(C) an organoaluminum compound; and
(D) an aromatic hydrocarbon solvent comprising C₉ substituted benzenes; wherein:
the aromatic hydrocarbon solvent comprises at least 75 wt. % C₉ substituted benzenes and/or from 5 to 25 wt. % C₁₀+ substituted benzenes; and
the C₉ substituted benzenes comprise a trimethyl benzene, a methyl ethyl benzene, or any combination thereof.
